(19) United States
(12) Patent Application Publication
(10) Pub. No.: US 2026/0335124 A1
(43) Pub. Date: Oct. 29, 2026

(54) HEAT TRANSFER DEVICE, APPARATUS FOR THERMAL THERAPY TREATMENT AND METHOD

(71) Applicant: HILOTHERM HOLDING AG, Oberwil bei Zug (CH)

(72) Inventor: Christian Stegmann, Burgberg im Allgäu (DE)

(21) Appl. No.: 19/387,942

(22) Filed: Nov. 13, 2025

(30) Foreign Application Priority Data
Nov. 15, 2024 (LU) 103454

(51) Int. Cl. A61F 7/00

(52) U.S. Cl. CPC A61F 7/0053; A61F 2007/0036; A61F 2007/0056

(57) ABSTRACT

A heat transfer device can be brought into contact with a hand of a human to cool the hand or transfer heat to the hand. The heat transfer device is for use with a heat transfer fluid arranged to flow through a fluid chamber of the heat transfer device. A front portion of the heat transfer device is arranged to cover at least a portion of the fingers of the hand on the back of the hand, wherein the heat transfer device comprises a rear portion which extends in a proximal direction from the front portion, wherein the rear portion has an opening in the region of the opisthenar area so as to leave at least a portion of the opisthenar area uncovered. An apparatus for use in the thermal therapy treatment of a human hand, in particular for use in the treatment of tissue damage after surgical interventions, of injuries, of inflammations and of chronic diseases of the rheumatic type, and in particular for assisting chemotherapeutic treatments, comprises such a heat transfer device and a temperature control device for controlling a temperature of the heat transfer fluid. The temperature control device is connected, in a fluid conducting manner, to the heat transfer device and is further arranged to temper the heat transfer device with the heat transfer fluid flowing through the heat transfer device.

### CROSS REFERENCE TO RELATED APPLICATIONS

This application claims priority to Luxembourg Patent Application Serial No. 103454 filed on Nov. 15, 2024, the disclosure of which is hereby incorporated by reference in its entirety for all purposes.

The present invention relates to a heat transfer device for use in the thermal therapy treatment of a hand of a human, and in particular for use in the treatment of tissue damage after surgical interventions, of injuries, of inflammations and of chronic diseases of the rheumatic type, and in particular for assisting chemotherapeutic treatments. The invention extends to an apparatus comprising such a heat transfer device and a temperature control device, as well as to a method of operating such a heat transfer device and such an apparatus.

In the context of the present invention, the term “thermal therapy treatment” is intended to be understood to mean a treatment in which heat is supplied to a human hand, or removed therefrom.

WO 2022/101320 A1 discloses a heat transfer device and an apparatus of the type described above. The entire content of WO 2022/101320 A1 is incorporated herein by reference. Briefly, at least one heat transfer device disclosed in this document comprises a temperature control fluid chamber formed by first and second flexible material layers. The first and second flexible material layers together can be folded over, and a human hand can be accommodated between the folded-over flexible material layers so that the temperature control fluid chamber substantially completely surrounds the human hand. A temperature control fluid can flow through the temperature control fluid chamber so as to heat or cool the hand. The heat transfer device disclosed in this document can be used together with a temperature control device, for example a temperature control device also disclosed in this document (the temperature control device and the heat transfer device together forming the apparatus for thermal therapy treatment), whereby the temperature control device causes the temperature control fluid to flow through the heat transfer device. To this end, the heat transfer device can be connected to the temperature control device via hoses or similar to convey the temperature control fluid between the temperature control device and the heat transfer device.

Whilst the heat transfer device and the apparatus disclosed in this document work very well for their intended purpose, the present inventor has perceived a need to develop this heat transfer device and apparatus further.

Accordingly, it is an object of the present invention to provide an alternative heat transfer device, apparatus and corresponding method, in particular an improved heat transfer device, apparatus and corresponding method.

Aspects of the present invention are set out in the claims.

A first aspect of the present disclosure provides a heat transfer device which is provided to be brought into contact with a hand of a human and which is arranged to transfer heat to, or from, the hand, the hand being attached to the end of an arm at a wrist and having a first main side and a second main side located opposite the first main side, the first main side comprising the opisthenar area of the hand and the second main side comprising the palm of the hand, wherein a distal direction of the hand is defined as a direction towards the fingertips of the hand, and a proximal direction of the hand is defined as a direction towards a wrist, the distal direction and the proximal direction together defining a longitudinal direction, and a direction perpendicular to the longitudinal direction and generally along the surface of the hand being a widthwise direction, wherein the heat transfer device is for use with a heat transfer fluid arranged to flow through the heat transfer device,
wherein the heat transfer device comprises a fluid chamber through which the heat transfer fluid is arranged to flow;
wherein the heat transfer device comprises a front portion which is arranged to cover at least a portion of the fingers of the hand on the first main side of the hand,
wherein the heat transfer device comprises a rear portion which extends in the proximal direction from the front portion, wherein the rear portion has an opening in the region of the opisthenar area so as to leave at least a portion of the opisthenar area uncovered.

By leaving at least a portion of the opisthenar area uncovered, the heat transfer device can be used in situations where there is a requirement for such a portion of the opisthenar area to remain accessible. For example, in some situations a portion of the opisthenar area needs to remain visible, for example so that changes of the opisthenar area such as swelling, change of color, bleeding etc. can immediately be detected. In some situations, a portion of the opisthenar area needs to be (easily) accessible, for example for inserting a hypodermic needle into the opisthenar area.

The inventor has found that, in applications where cooling of the hand is required, e.g. in order to reduce or prevent polyneuropathy during chemotherapy, it may be sufficient to cool certain regions of the hand whilst not (directly) cooling a portion of the opisthenar area by leaving a portion of the opisthenar area uncovered, i.e. not in contact with the heat transfer device. Firstly, polyneuropathy (primarily) affects the fingers and not (so much) the opisthenar area of the hand, and therefore (primarily) cooling the fingers may be sufficient to reduce polyneuropathy to a suitable degree (or preventing it altogether). Secondly, if the fingers are cooled by the heat transfer device, the opisthenar area is indirectly cooled as well, e.g. by the circulation of (cooled) blood from the fingers to the opisthenar area. Thirdly, if the palm (on the second side) of the hand is cooled as well, this may also cool the opisthenar area (e.g. through convection or conduction).

Corresponding considerations apply in the context of applications where heating of the hand is required.

In the interest of a compact disclosure, in the following, aspects and embodiments of the present disclosure will be explained in relation to cooling a hand, whereby it will be understood that aspects and embodiments of the present disclosure can equally be used in relation to heating a hand. Further, the reduction or prevention of polyneuropathy will be used, by way of example only, as a possible application, whereby it will be understood that embodiments of a heat transfer device, apparatus and method disclosed herein can equally be used in the context of other applications of cooling or heating a hand.

In the context of the present disclosure, reference is made to the hand of a human. Clearly, different people have different size hands, and the shape of their hands may also vary. Accordingly, several sizes of heat transfer devices may be provided in order to ensure that a heat transfer device is available for a relatively large range of hand sizes/shapes, in particular not only for adults but also (young) children or even babies. However, the inventor envisages that an exact fit between heat transfer device and hand is not essential, in particular if the material of the heat transfer device has a suitable degree of flexibility to adapt to the size and shape of the hand of the user. Further, the inventor envisages that the size and/or shape of a human hand can be taken into account during manufacture of a heat transfer device so that, for a heat transfer device of a particular size/shape, the opening will be in a suitable location and will be of a suitable size in relation to the size/shape of the hand for which the heat transfer device is intended, and in particular such that the opening will be in the region of the opisthenar area of the respective hand. In any event, it may be possible to cover a given range of hand sizes/shapes with only a small number of different models of heat transfer devices, e.g. three of five different models to cover the entire range of hands from babies to adults. The inventor envisages that a single model of heat transfer device will be suitable for the hands of at least 90% or at least 99% of all adults.

In the following, various embodiments of the heat transfer device will be described, by way of example, each of which, unless expressly excluded or technically impossible, can be combined as desired with one another and with the other aspects of the present disclosure.

The opening may have a size:
such that the portion of the opisthenar area left uncovered is large enough to accommodate a hypodermic needle, preferably also an adhesive bandage for temporarily fixing the hypodermic needle to the hand, and/or
such that the portion of the opisthenar area left uncovered has at least a minimum width in the widthwise direction, wherein the minimum width is at least 3 mm, 5 mm, 1 cm, 2 cm, 3 cm or 4 cm, and/or such that the portion of the opisthenar area left uncovered has at most a maximum width in the widthwise direction, wherein the maximum width is at most 6 cm, 5 cm, 4 cm, 3 cm or 2 cm.

In the lengthwise direction, the opening may have a minimum length, wherein the minimum length is at least 5 mm, 1 cm, 2 cm, 3 cm, 4 cm or 5 cm, for example.

One way of determining a suitable size/location for the opening is as follows. We take a representative sample (in terms of size and/or shape) of hands of human adults and determine an average human adult hand therefrom. We then manufacture a heat transfer device which would accommodate this average human adult hand. We then determine the size and location of the opisthenar area of that hand and provide an opening in the heat transfer device so that the opening is in the region of the opisthenar area, whereby there is typically no need for the entire opisthenar area to remain uncovered. If we then want to manufacture a heat transfer device for a different size and/or shape hand, in particular wider or narrower or longer or shorter, we stretch or reduce all dimensions of the heat transfer device in the lengthwise and/or widthwise direction as required, including the dimensions of the opening. The inventor expects that the opening for the modified heat transfer device will still be in a suitable location and of a suitable size in relation to the hand for which the modified heat transfer device is intended since the dimensions will have changed proportionally.

The opening may be open towards the proximal direction.

In the case of an opening that is open towards the proximal direction, the material of the heat transfer device does not entirely surround the opening on the first main side. By way of example, this can be particularly useful if a hypodermic needle is to be inserted into the opisthenar area of the hand before the heat transfer device is applied to the hand since no portion of the material of the heat transfer device needs to be slid over the hypodermic needle (or similar).

For the avoidance of doubt, in the present context, an “opening of the heat transfer device that is open towards the proximal direction” is not to be seen as analogous to the opening of e.g. a typical glove through which a hand is introduced into the glove since the location of the opening of the heat transfer device is intended to be in the region of the opisthenar area, which is not the case for the opening of a typical glove.

In some embodiments, the heat transfer device does not have a cover for covering the opening. The absence of a cover means that, in normal use of the heat transfer device, the opening stays open (and the portion of the opisthenar area remains visible through the opening).

The rear portion may comprise at least one lateral portion which is arranged to cover a portion of the opisthenar area, wherein the lateral portion is adjacent to the opening in the widthwise direction.

In this way it is possible for at least a portion of the opisthenar area (where the lateral portion is located) to be cooled via the heat transfer device whilst another portion of the opisthenar area remains uncovered (where the opening is located). This may further enhance the cooling performance of the heat transfer device. The rear portion may in particular comprise two such lateral portions, one on each side of the opening in the widthwise direction, thereby improving the cooling performance even further. The lateral portion or portions may in particular extend to a location approximately corresponding to the wrist of the hand.

The front portion may be arranged to cover substantially the entire finger region on the first main side of the hand.

In this context, the term “entire finger region” can, for example, be understood to refer to the entire region of the four fingers, i.e. index finger to little finger, excluding the thumb. Covering substantially the entire finger region with the heat transfer device can ensure particularly effective cooling of the fingers.

The heat transfer device may be arranged to cover at least a portion of the hand on the second main side, in particular at least 50%, 60%, 70%, 80% or 90%, in particular substantially all of the hand on the second main side.

The part of the heat transfer device that is arranged to contact the second main side of the hand will be referred to as the bottom portion, whereas the part of the heat transfer device that is arranged to contact the first main side of the hand will be referred to as the top portion.

In general, the higher the proportion of coverage, the better the cooling performance of the heat transfer device. Further, the material of the heat transfer device of the bottom portion may provide the heat transfer device with more mechanical stability. In addition, flexible bands or similar may be provided to enable the top portion of the heat transfer device to be connected, in particular detachably connected, to the bottom portion. The bands or similar can be (substantially permanently) connected to one of the top portion and the bottom portion of the heat transfer device. The bands or similar could be provided with hooks or loops of a hook-and-loop fastener system, or similar. This would be particularly suitable for a heat transfer device which can be opened so as to allow a hand (for example the second main side of the hand) to be placed on e.g. the bottom portion of the heat transfer device and the top portion of the heat transfer device to be folded over the hand so that the hand is surrounded by the heat transfer device on both main sides. Alternatively, the top portion and the bottom portion of the heat transfer device can be (substantially permanently) connected on two lateral sides (the side of the thumb and the side of the little finger), in which case the heat transfer device would normally be slid over the hand with a movement in the proximal direction.

On the second main side of the hand and in the proximal direction, the heat transfer device may be arranged to extend beyond the wrist.

This may further enlarge the surface available for cooling the hand (or the wrist and perhaps part of the arm).

The heat transfer device may further comprise a member arranged to extend around the arm to secure the heat transfer device to the arm.

This can help to prevent the heat transfer device from (accidentally) sliding off the hand. Further, the member may provide a surface on which to attach a fluid line leading to the hypodermic needle. This can help to prevent the hypodermic needle from accidentally being detached from the hand, for example as a result of a movement of the hand or arm.

Again, the member may comprise a band or similar, in particular a band or similar that is (substantially permanently) attached to the heat transfer device at one end and which can be releasably connected to the heat transfer device at its other end. This band or similar could again be provided with hooks or loops of a hook-and-loop fastener system, or similar.

The member may at least partially be see-through.

This can enable any changes of the skin under the member to be detected quickly, e.g. swelling, bleeding or changes in the color of the skin.

The heat transfer device can have at least a first fluid connection to enable the heat transfer fluid to enter the fluid chamber and at least a second fluid connection to enable the heat transfer fluid to exit the fluid chamber.

The first and second fluid connections can, for example, be ports to which a hose or similar can (releasably) be connected. Alternatively, a hose or similar can substantially permanently be connected to the heat transfer device.

The heat transfer device can also have more than two fluid connections, for example three or four fluid connections. For example, the fluid chamber can be subdivided into two partial chambers, which may or may not be interconnected in terms of an exchange of fluid. Each partial fluid chamber may have its own (external) fluid connection for the heat transfer fluid to enter the respective partial fluid chamber and an (external) fluid connections for the heat transfer fluid to exit the respective partial fluid chamber. Alternatively, the partial fluid chambers may be interconnected in terms of exchange of fluid, whereby the (overall) fluid chamber may have, for example, one input connection and two output connections for the heat transfer fluid, or two input connections and one output connection for the heat transfer fluid.

A second aspect of the present disclosure provides an apparatus for use in thermal therapy treatment of a human hand, in particular for use in the treatment of tissue damage after surgical interventions, of injuries, of inflammations and of chronic diseases of the rheumatic type, and in particular for assisting chemotherapeutic treatments, wherein the apparatus comprises:
a temperature control device which is provided in order to control a temperature of a heat transfer fluid; and
at least one heat transfer device as any one described above,
wherein the temperature control device is connected, in a fluid conducting manner, to the at least one heat transfer device, and is further arranged to temper the at least one heat transfer device with the heat transfer fluid flowing through the heat transfer device.

As used herein, the term “control device” is intended to be understood mean a device which controls (without feedback) and/or a device which controls with feedback, other devices on the basis of data or values supplied to it and/or data or values or settings entered by a user. Similar considerations apply with respect to the terms “to control”, “controlling” and the like.

A third aspect of the present disclosure provides a method of using any one of the heat transfer devices described above, or any one of the apparatuses for thermal therapy treatment of a hand of a human as described above, wherein the method comprises:
applying the heat transfer device to a hand of a human; and
causing the heat transfer fluid to flow through the heat transfer device.

The above actions can be carried out in the stated order or a different order. For example, it is possible to start the fluid flow (i.e. causing the heat transfer fluid to flow through the heat transfer device) before applying the heat transfer device to the hand.

The method of using a heat transfer device or an apparatus as described above can either be used in the context of a therapeutic treatment, or alternatively for non-therapeutic purposes.

The terms “encompasses”, “contains”, “includes”, “comprises”, “has”, “with”, as they may be used herein, or any other variation of these, are intended to cover an inclusion which is not exclusive. Accordingly, for example, a method or a device which encompasses or comprises a list of elements is not necessarily limited to those elements, but may include other elements which are not explicitly listed or which inherently form part of such a method or device.

Further, unless expressly stated to the contrary, “or” refers to an inclusive “or” and not to an exclusive “or”. For example, a condition “A or B” is satisfied by any one of the following conditions: A is true (or present) and B is false (or not present), A is false (or not present) and B is true (or present), and both A and B are true (or present).

The terms “a” or “an”, as they are used herein, are defined in the sense of “at least one”. The terms “another” and “a further” as well as any other variant thereof are to be understood in the sense of “at least one further”.

The term “a plurality of”, as it may be used herein, as well as any other variant thereof, is to be understood in the sense of “two or more”.

The terms “arranged to”, “configured to” or “set up to” fulfil a particular function (and variations of these terms, respectively), as they may be used herein, are intended to be understood to mean that the respective device or component thereof is already present in a configuration or with a setting in such a way that it can perform the function, or that it can at least be adjusted—i.e. that it can be configured—to perform the function after appropriate adjustment. In this context, the configuration can be carried out, for example, by a corresponding setting of parameters of a process sequence or of switches or the like, for activating or deactivating functionalities and/or settings. In particular, the device can have several predetermined configurations or modes of operation, so that the configuring can be carried out by selecting one of these configurations or modes of operation.

Further advantages, features and applications of the present invention are described in the following detailed description and the appended figures, wherein:

FIG. 1 schematically shows a top view of a right-hand arm and hand of a human.

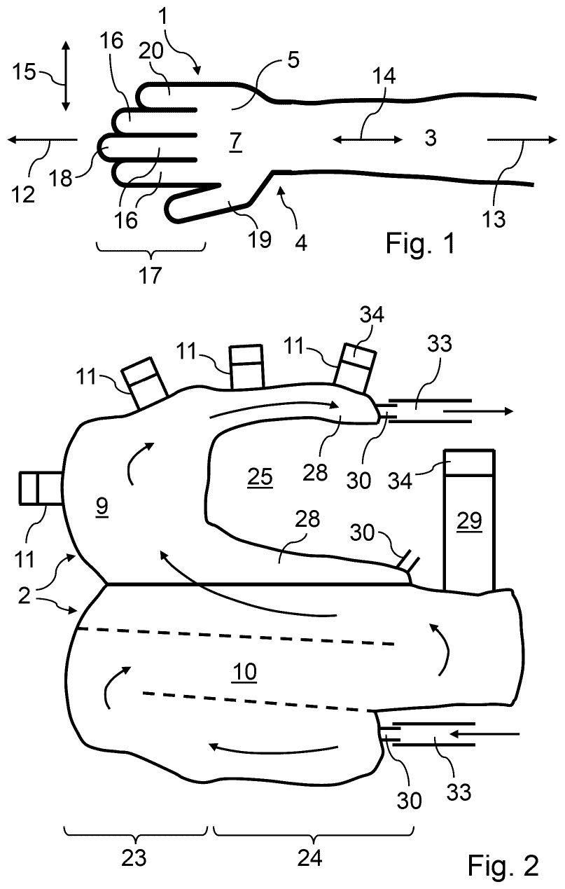

FIG. 2 schematically shows a top view of a heat transfer device in accordance with an embodiment of the present disclosure in an unfolded condition.

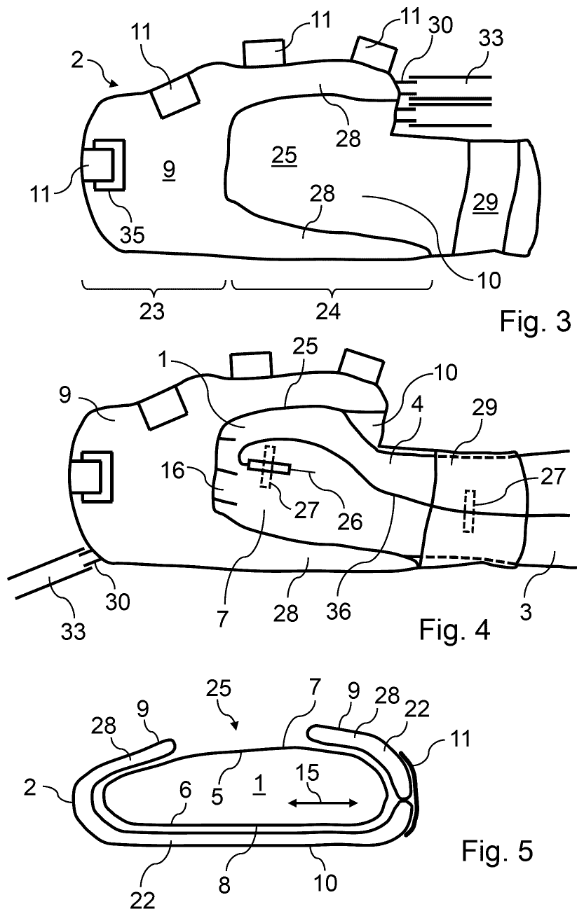

FIG. 3 schematically shows a top view of a heat transfer device in accordance with an embodiment of the present disclosure in a folded condition.

FIG. 4 schematically shows a top view of a heat transfer device in accordance with an embodiment of the present disclosure in a folded condition, with a hand of a human introduced into the heat transfer device.

FIG. 5 schematically shows a cross-sectional view of a heat transfer device in accordance with an embodiment of the present disclosure in a folded condition, with a hand of a human introduced into the heat transfer device.

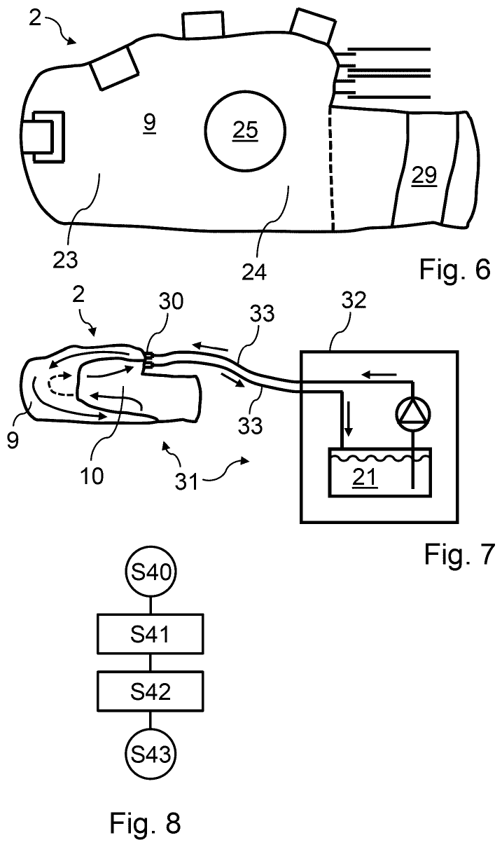

FIG. 6 schematically shows a top view of a heat transfer device in accordance with an embodiment of the present disclosure in a folded condition.

FIG. 7 schematically shows an apparatus for use in the thermal therapy treatment of a human hand in accordance with an embodiment of the present disclosure.

FIG. 8 shows a flowchart illustrating a method in accordance with an embodiment of the present disclosure.

In the figures, identical reference signs are used for the same or similar elements of the embodiments of the disclosure. To the extent that an element is shown in more than one figure, this element and its function will not necessarily be explained repeatedly.

FIG. 1 schematically shows a top view of a right-hand arm 3 and hand 1 of a human. The hand 1 is attached to the end of the arm 3 at a wrist 4. The hand 1 is that of a typical human with a thumb 19, as well as four fingers 16, including little finger 20. Human hands with more, or fewer, fingers 16, or without thumb 19, are also to be regarded as a human hand in accordance with the present disclosure.

As used in the present disclosure, a distal direction 12 is defined as a direction towards the fingertips 18 (only the fingertip 18 of the middle finger is labelled in FIG. 1). The proximal direction 13 is defined as a direction in the opposite direction with respect to the distal direction 12. Together, the proximal direction 13 and the distal direction 12 also define longitudinal direction 14. Perpendicular to the longitudinal direction 14, and generally along the surface of the hand 1, is a widthwise direction 15.

The side of the hand 1 that is shown in FIG. 1 is referred to herein as the first main side 5 of the hand 1. This includes the opisthenar area 7, generally between the finger region 17 and the wrist 4. The opisthenar area 7, or the first main side 5 of the hand 1 is sometimes also referred to as the back of the hand 1.

The second main side 6 of the hand 1 opposite the first main side 5 includes the palm 8 of the hand 1. Neither the second main side 6 nor the palm 8 are shown in FIG. 1. Their location is indicated in FIG. 5.

FIG. 2 schematically shows a top view of a heat transfer device 2 in accordance with an embodiment of the present disclosure in an unfolded condition. The heat transfer device 2 of FIG. 2 would be suitable for a right hand. The heat transfer device 2 can generally be constructed as has been disclosed in WO 2022/101320 A1. In particular, any features of the heat transfer device 2 disclosed in this document can also be features of a heat transfer device 2 of the present disclosure, unless the present disclosure specifies otherwise. Generally speaking, the heat transfer device 2 has two portions of somewhat flexible material, a top portion 9 and a bottom portion 10. These are joined along the folding line or folding region shown as a horizontal line approximately in the middle of FIG. 2. The heat transfer device 2 can be folded or bent along the folding line or folding region so that the top portion 9 generally comes to rest on the bottom portion 10.

It is to be noted that directional terms such as “top” and “bottom”, as used in the expressions top portion 9 and bottom portion 10, are used for illustration purposes only, and the skilled person will understand that the heat transfer device 2 can be used in orientations other than those described, for example in an orientation in which the bottom portion 10 is located above the top portion 9. Further, directional terms may be used in order to refer to features as they are shown in the drawings (e.g. “the fingertips 18 are on the left” in FIG. 1), and this is also not to be understood as a limitation.

Referring again to FIG. 2, the heat transfer device 2 comprises at least two flexible layers which are attached (e.g. fused) to one another along their periphery (the periphery of top portion 9 and bottom portion 10) in order to define a fluid chamber 22 (see FIG. 5) therebetween. The fluid chamber 22 is a substantially fluid tight enclosure, except that it has at least two fluid connections 30. Three fluid connections 30 are shown in FIG. 2, whereby we first consider an embodiment in which only the fluid connections 30 towards the top and bottom of the figure are present (i.e. an embodiment where the fluid chamber 22 of the heat transfer device 2 is sealed at the location of the fluid connection 30 shown in the middle of FIG. 2).

A heat transfer fluid 21 (see FIG. 7) can be introduced into the fluid chamber 22 through fluid connection 30 via fluid line 33 shown in the bottom right-hand corner of the FIG. 2. Fluid line 33 can be a flexible tube or hose for conveying a fluid. Whilst a small gap is shown between fluid line 33 and fluid connection 30 for illustration purposes, the fluid line 33 is (removably) attached to fluid connection 30 in a substantially fluid tight manner. In an alternative embodiment, fluid line 33 is formed integrally with fluid connection 30 or with the heat transfer device 2 and is therefore substantially permanently attached to the heat transfer device 2.

The flow of heat transfer fluid 21 into, through and out of the heat transfer device 2 is generally indicated by arrows. The heat transfer fluid 21 follows a generally serpentine or labyrinthine path, first through bottom portion 10 and then through top portion 9, before exiting the heat transfer device 2 via fluid connection 30 shown in the top right-hand corner of FIG. 2. The heat transfer fluid 21 having exited the heat transfer device 2 can then flow through another fluid line 33. The fluid connection 30 and the fluid line 33 shown in the top right-hand corner of FIG. 2 can be constructed in a similar or a different manner to the fluid connection 30 and fluid line 33 shown in the bottom right-hand corner of the FIG. 2. The same applies to the fluid connection 30 in the middle of FIG. 2, where provided.

Two dashed lines are shown, by way of example, in the bottom portion 10 of the heat transfer device 2. The flexible layers forming the fluid chamber 22 can be attached (e.g. fused) to one another along these dashed lines in order to define the serpentine path through the heat transfer device 2. Other arrangements of such fusing lines are possible to define other patterns of the flow path. Likewise, heat transfer devices 2 without any such fusing lines are possible—this might be appropriate in the case of a relatively small heat transfer device 2, e.g. for small children or babies. The two flexible layers forming the fluid chamber 22 may also be attached to one another to form individual “islands”, as has been described in WO 2022/101320 A1. Such fusing lines and/or individual islands may help to generally fix the position of the flexible layers relative to one another, not only at the periphery of the heat transfer device 2 but also over a wider area.

Whilst a horizontal (folding) line in the middle of FIG. 2 is used to visually separate the top portion 9 from the bottom portion 10, the top portion 9 and the bottom portion 10 are in fluid communication with one another, either in one or more individual locations along this folding line, or along the entire folding line.

A front portion 23 and a rear portion 24 of the heat transfer device 2 are shown in FIG. 2. The front portion 23 is located towards the distal end of the heat transfer device 2, whereas the rear portion 24 is located towards the proximal end of the heat transfer device 2. In the bottom portion 10 of the heat transfer device 2, the front portion 23 and rear portion 24 form a substantially continuous surface, without any openings (although, in alternative embodiments, openings could be provided in the bottom portion 10). In the top portion 9 of the heat transfer device 2, the front portion 23 forms a substantially continuous surface, without any openings. By way of contrast, the rear portion 24 of the top portion 9 has an opening 25. Two lateral portions 28 are part of the rear portion 24 of the top portion 9 of the heat transfer device 2. Starting from the front portion 23, the lateral portions 28 extend in the proximal direction 13 and laterally (i.e. in the widthwise direction 15) surround the opening 25. The opening 25 is open towards the proximal direction 13. The front portion 23 and the lateral portions 28 of the top portion 9 together generally form a crescent or sickle shape. In alternative embodiments, only one lateral portion 28 is provided as part of the top portion 9, or none at all.

As will be explained in more detail below, a hand 1 (of the right arm) can be placed on the bottom portion 10. The top portion 9 can then be folded over, along the folding line, so that it comes to rest on the hand 1. In order to secure the top portion 9 to the bottom portion 10, the heat transfer device 2 is provided with one or more fasteners or closure members 11. These can be used to temporarily (and releasably) secure the top portion 9 with respect to the bottom portion 10. The fasteners 11 can, for example, comprise a strip or band of fabric material or plastics material. The fasteners 11 can, for example, be (substantially permanently) attached to the top portion 9, e.g. sewn onto the top portion 9. The fasteners 11 can form part of a hook and loop fastener (sometimes also referred to as Velcro®) or similar. To this end, the loose end of the fasteners 11 can be provided with an adhesive element 34, for example the hook element 34 of a hook and loop fastener. When the top portion 9 is folded onto the bottom portion 10 (with a hand 1 placed therebetween) the fasteners 11 can be folded onto the outer surface of the bottom portion 10 in order to (temporarily) secure the top portion 9 to the bottom portion 10, as will be described in more detail below. Alternatively, the fasteners 11 can be permanently secured to the bottom portion 10 and would then temporarily be secured to the top portion 9. In the example of FIG. 2, the heat transfer device 2 is provided with four such fasteners 11, but more, or fewer, fasteners 11 can be provided. The adhesive element 34 of fasteners 11 need not be a hook element of a hook and loop fastener but could also be provided with a surface having a releasable adhesive provided thereon. Further, other fastening arrangements are possible, including fasteners which are provided completely separately from the heat transfer device 2 and which are temporarily attached both to the bottom portion 10 and the top portion 9, or an arrangement with a strap and buckle, or elastic bands placed around the heat transfer device 2 when in the folded condition.

The bottom portion 10 extends further in the proximal direction 13 than the top portion 9. It projects beyond a longitudinal position where, typically, the wrist 4 of a user would be located when the hand 1 of the user is placed in the heat transfer device 2. A further fastener 29 is attached to the proximally projecting portion (not labelled) of the bottom portion 10. The fastener 29 can be constructed generally in a similar way as the fasteners 11. However, the fastener 29 in this example is a member 29 which, at least for the most part, is see-through. It is also longer than the fasteners 11 since it is intended to extend around the wrist 4 or the distal end of arm 3 of the user to secure the heat transfer device 2 to the arm 3. The see-through member 29 can again be provided with an adhesive element, such as a hook element of a hook and loop fastener, so that the (in FIG. 2 upper) end of see-through member 29, after being placed around the wrist 4 of the user, can temporarily be attached to the bottom portion 10.

The fluid chamber 22 can extend into the region of the proximally projecting portion of the bottom portion 10 so that the heat transfer fluid 21 would also flow through that region. Alternative embodiments are possible in which the fluid chamber 22 does not extend into the proximally projecting portion of the bottom portion 10. To this end, the two flexible layers making up the fluid chamber 22 could be attached (e.g. fused) together, at least along a line at the distal end of the proximally projecting portion, for example at a position in the longitudinal direction 14 at the proximal end of the lateral portion 28 in the middle of FIG. 2.

FIG. 3 schematically shows a top view of a heat transfer device 2 in accordance with an embodiment of the present disclosure in a folded condition. The heat transfer device 2 of FIG. 3 can be the same as, or substantially similar to, the heat transfer device 2 shown in FIG. 2, so that most parts of the description of FIG. 2 also apply to FIG. 3. Accordingly, the description of FIG. 3 will not repeat corresponding features already described in connection with FIG. 2. The same applies to FIGS. 4 to 7.

The heat transfer device 2 shown in FIG. 3 would be suitable for the left hand 1 of a user. In the folded condition of the heat transfer device 2, the fluid connections 30, which were near the top and bottom in FIG. 2, are now close to each other. For example, what is shown in FIG. 3 as the topmost fluid connection 30 is attached to lateral portion 28 of top portion 9, and the other fluid connection 30 (not labelled) in FIG. 3 is attached to the bottom portion 10.

The see-through member 29 is shown as extending across the proximally projecting portion of bottom portion 10. Of the four fasteners 11 shown in FIG. 2, the two leftmost ones are folded over in FIG. 3, whereas the two topmost ones have not (yet) been folded over. Further, FIG. 3 illustrates an example where the fasteners 11 are permanently attached to the bottom portion 10 and where, after being folded over, they are releasably attached to the top portion 9.

In the case of hook and loop fasteners 11, the outer material of that portion to which the fastener 11 is intended to be releasably attached (top portion 9 in FIG. 3, or bottom portion 10 in FIG. 2), can be such that the hook element 34 can be temporarily attached directly to that outer material, as might be the case for the three topmost fasteners 11 shown in FIG. 3. Alternatively, a loop element 35 of a hook and loop fastener can be (permanently) attached to the outer surface of the corresponding portion (9 or 10) so that a hook element 34 can temporarily be attached to this loop element 35, as is the case for the leftmost fastener 11 in FIG. 3.

FIG. 4 schematically shows a top view of a heat transfer device 2 in accordance with an embodiment of the present disclosure in a folded condition, with a (left) hand 1 of a human introduced into the heat transfer device 2.

In the embodiment shown in FIG. 4, the fluid connections 30 are attached to the front portion 23 (near what would be the region of the fingertips 18), rather than the rear portion 24. Both fluid connections 30, respectively for the top portion 9 and the bottom portion 10 of the heat transfer device 2 are located near the folding line, which means that they are close to one another both in the folded and the unfolded condition of the heat transfer device 2, which may be convenient in terms of handling the fluid lines 33, in particular when the heat transfer device 2 is being applied to a hand 1. In FIG. 4, only one fluid connection 30 and fluid line 33 is shown—the other fluid connection 30 and fluid line 33 would be hidden behind the one that is shown.

Other configurations for the fluid connections 30 and fluid lines 33 are possible. One such alternative configuration is shown in FIG. 2, where three fluid connections 30 are shown, including the fluid connection 30 in the middle of FIG. 2. In this case, a fluid line 33 would also be attached to the fluid connection 30 in the middle of the figure (not shown). The fluid connection 30 at the top and in the middle of FIG. 2 could then both serve as fluid outlets. For example, the flexible layers constituting the fluid chamber 22 could, along much of the (horizontal) folding line in the middle of FIG. 2, be fused together, leaving only a relatively small fluid passage across the folding line, for example near the distal end of the folding line. The heat transfer fluid 21 flowing through the bottom portion 10 would then be forced to flow all the way to the distal end of bottom portion 10, then flow through the fluid passage located towards the distal end in order to enter the top portion 9. There, part of the heat transfer fluid 21 would flow towards the fluid connection 30 in the middle of the figure, and another part would flow towards the fluid connection 30 at the top of the figure. However, this is merely one example of possible configurations of the fluid connections 30 and the flow path. Another example might have (only) two fluid connections 30 close to proximal end of the folding line in the middle of FIG. 2, one attached to the top portion 9 and one attached to the bottom portion 10—which would again mean that both fluid connections 30 are close to one another both in the folded and the unfolded condition of the heat transfer device 2. A possible flow path arrangement would then guide the heat transfer fluid 21 along much or all of the bottom portion 10, then through a passage at the folding line, then along much or all of the top portion 9, and finally to the outlet (fluid connection 30 attached to the top portion 9).

One skilled in the art will appreciate that, in all embodiments described herein, the direction of fluid flow could be reversed so that the heat transfer fluid 21 would flow in the opposite direction to what is described and/or shown in the drawings.

Returning to FIG. 4, a hand 1 is shown as having been placed between the top portion 9 and the bottom portion 10. Much or all of the finger region 17 is covered or surrounded both by the top portion 9 and the bottom portion 10. Only a small portion of the fingers 16, at the proximal end of fingers 16, is shown as not having been covered by top portion 9. The opening 25 reveals much of the opisthenar area 7. As far as the top portion 9 is concerned, only the lateral portions 28 cover parts of the first main side 5 of the hand 1. Therefore, the heat transfer device 2 is in contact with the hand 1 all along the second main side 6 of the hand 1 and, on the first main side 5 of the hand 1, in the finger region 17 and the lateral portions 28, and (direct) cooling can take place in these regions. As has already been described above, no direct cooling of the first main side 5 of the hand 1 takes place in the region of the opening 25.

A hypodermic needle 26—typically comprising a metal needle and, attached thereto, a plastic part—is shown as having been inserted in the opisthenar area 7. The plastic part can be secured to the skin of the opisthenar area 7 with an adhesive bandage or sticky plaster 27 or similar. As the heat transfer device 2 is formed with the opening 25, the area where the hypodermic needle 26 and, if applicable, adhesive bandage 27 are located is left free so that this area remains visible during use of the heat transfer device 2 (i.e. when the hand 1 is accommodated in the heat transfer device 2). A fluid line (or tube or hose) 36 can be connected to hypodermic needle 26 to supply fluids into the hand 1 in the region of the opisthenar area 7, or to remove fluids therefrom.

The see-through member 29 at the proximal end of the heat transfer device 2 is shown as having been placed over wrist 4 or distal end of arm 3. See-through member 29 is shown somewhat wider in FIG. 4 than in FIGS. 2 and 3. In the region of the see-through member 29, the outline of wrist 4 or arm 3 of the human is indicated by dashed lines towards the top and bottom of the figure. As the see-through member 29 is at least partly transparent, the wrist 4 and/or arm 3 of the human can be monitored, even where it is covered by see-through member 29. As the opisthenar area 7 is also not covered by opening 25, much of the opisthenar area 7 as well as the entire wrist 4 and lower arm 3 of the human remains visible and can be monitored.

The see-through member 29 can serve as a surface onto which an adhesive bandage or sticky plaster 27 can be attached, for example so as to (temporarily) secure the fluid line 36 to the see-through member 29.

When a heat transfer device 2 in accordance with the embodiments of FIG. 3 or 4 is used, a hypodermic needle 26 can first be inserted into the opisthenar area 7 (and, if applicable, an adhesive bandage 27 can be attached thereto), and the hand 1 can then be inserted into the heat transfer device 2 (or the heat transfer device 2 can be applied to the hand 1) without the top portion 9 touching, or sliding over, the hypodermic needle 26 (and adhesive bandage 27). This is because the opening 25 is open towards the proximal direction. Once the hand 1 is accommodated in the heat transfer device 2, the see-through member 29 can be placed over the wrist 4 or lower arm 3. Further, the hypodermic needle 26 can be removed from the opisthenar area 7 while the hand 1 remains accommodated in the heat transfer device 2. Conversely, it is also possible to remove the heat transfer device 2 from the hand 1 while the hypodermic needle 26 remains attached to the opisthenar area 7. Merely the see-through member 29 would need to be opened to enable the heat transfer device 2 to be removed from the hand 1.

FIG. 5 schematically shows a cross-sectional view of a heat transfer device 2 in accordance with an embodiment of the present disclosure in a folded condition, with a hand 1 of a human introduced into the heat transfer device 2. FIG. 5 represents a cross-sectional view in the widthwise direction 15, i.e. the drawing plane is perpendicular to the longitudinal direction 14. As regards the longitudinal direction 14, the cross-sectional view of FIG. 5 could be at a longitudinal position where the needle 26 enters the opisthenar area 7 in FIG. 4.

In FIG. 5, the opisthenar area 7 on the first main side 5 of the hand 1 is located towards the top, and the palm 8 on the second main side 6 of the hand 1 is located towards the bottom. The second main side 6 of the hand 1 rests on bottom portion 10 of the heat transfer device 2 while the first main side 5 of the hand 1 is covered by the top portion 9 of the heat transfer device 2, except for the area left uncovered due to the opening 25. Accordingly, in the cross-sectional view of FIG. 5, only the lateral portions 28 cover the first main side 5 of the hand 1. The fastener 11 secures the top portion 9 and bottom portion 10 to one another. As shown in FIG. 5, the top portion 9 and bottom portion 10 have an outer layer and an inner layer forming the fluid chamber 22 therebetween. Whilst a small gap is shown between the hand 1 and the top portion 9 and bottom portion 10 for illustration purposes, the inventor envisages the heat transfer device 2 to be relatively tightly wrapped around the hand 1 so as to ensure that there is a suitable thermal contact between the heat transfer device 2 and the hand 1.

Whilst not shown in FIG. 5, the heat transfer device 2 can have further layers in addition to the two flexible layers forming the fluid chamber 22. For example, an inner (skin-facing) fabric layer may be provided on the inner side of the heat transfer device 2 in order to result in a more pleasant feeling to the human hand 1, in particular if the two flexible layers forming the fluid chamber 22 are made from plastics material. Further, an outer layer, which can again be a fabric layer, may be provided on the outer side of the heat transfer device 2. This outer fabric layer can in particular be such that a hook element 34 of fasteners 11 can be attached directly to that outer fabric layer. The heat transfer device 2 can also have a pressure unit (such as any one disclosed in WO 2022/101320 A1) on the outside of the fluid chamber 22 in order to press the fluid chamber 22 against the hand 1. The pressure unit can, for example, comprising an elastic material such as rubber foam supported, on the outside, by a support device, e.g. comprising a material that is less flexible than the elastic material. Instead of the elastic material, the pressure unit can comprise an inflatable bladder.

FIG. 6 schematically shows a top view of a heat transfer device 2 in accordance with an embodiment of the present disclosure in a folded condition. In many respects, the embodiment shown in FIG. 6 is similar to those of FIGS. 3 and 4. However, in FIG. 6, the opening 25 is not open towards the proximal direction 13. This may enable a larger surface of the first main side 5 of the hand 1 to be cooled whilst ensuring that a portion of the opisthenar area 7 remains visible (through the opening 25) and accessible, for example for a hypodermic needle 26. However, in contrast to the embodiments of FIGS. 3 and 4, removing the heat transfer device 2 from the hand 1 whilst leaving a hypodermic needle 26 (and, if applicable, also a fluid line 36) attached to the opisthenar area 7 may not be possible, or at least more difficult than in the case of the embodiments of FIGS. 3 and 4.

FIG. 6 indicates a further variation of the heat transfer device 2. In FIG. 6, a dashed line is shown at the proximal end of the rear portion 24, or at the transition between the rear portion 24 and the proximally projecting portion of the bottom portion 10. The proximally projecting portion of the bottom portion 10 (including the see-through member 29) could be omitted, i.e., in the proximal direction 13, the heat transfer device 2 would terminate at the position indicated by the dashed line. This might make it easier to slide the heat transfer device 2 on and off the hand 1.

In a further variation, which is not explicitly shown in the drawings, the top portion 9 would be permanently connected to the bottom portion 10 not only at the folding line, but along most parts of the periphery, i.e. also on the left and top of what is shown in FIGS. 3, 4 and 6. This means that the fasteners 11 would not be required. The heat transfer device 2 would then be open substantially only at the opening 25 and at the proximal end of the rear portion 24. In order to apply the heat transfer device 2 to a hand 1, the hand 1 could simply be slid into the heat transfer device 2.

FIG. 7 schematically shows an apparatus 31 for use in the thermal therapy treatment of a human hand 1 in accordance with an embodiment of the present disclosure.

The apparatus 31 comprises a heat transfer device 2 as any one described herein, a temperature control device 32 and at least two fluid lines 33 connecting the heat transfer device 2 to the temperature control device 32. The temperature control device 32 can be constructed in a similar way to any one disclosed in WO 2022/101320 A1, for example. Briefly, the temperature control device 32 shown in FIG. 7 has a storage tank for heat transfer fluid 21, as well as a pump represented by the symbol of a triangle in a circle. As indicated by the arrows within the temperature control device 32, along the fluid lines 33 and within the heat transfer device 2, heat transfer fluid 21 is pumped by the pump from the storage tank along one fluid line 33 leading to the heat transfer device 2. The heat transfer fluid 21 then flows through the heat transfer device 2 and, after leaving the heat transfer device 2 via fluid connection 30, flows through the second fluid line 33 back to the storage tank. The heat transfer fluid 21 in the storage tank is kept at a desired temperature by a heating or cooling unit (not shown).

FIG. 8 shows a flowchart illustrating a method in accordance with an embodiment of the present disclosure. After the start S40 of the method, a heat transfer device 2 is applied to the hand 1 of a human (S41). A heat transfer fluid 21 is then caused to flow through the heat transfer device 2 (S42). Thereafter, the method can end (S43).

While at least one example embodiment of the present invention has been described above, it has to be noted that a great number of variations thereto exist. Furthermore, it is to be appreciated that the described example embodiments only illustrate non-limiting examples of how the present invention can be implemented and that it is not intended to limit the scope, the application or the configuration of the apparatuses and methods described herein. Rather, the preceding description will provide the person skilled in the art with instructions for implementing at least one example embodiment of the invention, whereby it has to be understood that various changes in the functionality and the arrangement of the elements of the example embodiment can be made without deviating from the subject-matter defined by the appended claims and their legal equivalents. The features described herein may be combined with each other as desired, unless this is expressly excluded or technically impossible. Likewise, the features primarily described in connection with one aspect disclosed herein may correspondingly represent features of other aspects disclosed herein. In addition, any or all aspects and features disclosed herein are to be regarded as aspects of the present invention, either alone or in combination with other aspects and features disclosed herein.

### LIST OF REFERENCE SIGNS

1 hand
2 heat transfer device
3 arm
4 wrist
5 first main side of hand
6 second main side of hand
7 opisthenar area
8 palm
9 top portion of heat transfer device
10 bottom portion of heat transfer device
11 fastener
12 distal direction
13 proximal direction
14 longitudinal direction
15 widthwise direction
16 fingers
17 finger region
18 fingertips
19 thumb
20 little finger
21 heat transfer fluid
22 fluid chamber
23 front portion
24 rear portion
25 opening
26 hypodermic needle
27 adhesive bandage, sticky plaster
28 lateral portion
29 (see-through) member
30 fluid connection
31 apparatus for use in thermal therapy treatment
32 temperature control device
33 fluid line (tube, hose) for heat transfer fluid
34 adhesive element, hook element of hook and loop fastener
35 loop element of hook and loop fastener
36 fluid line (tube, hose) connected to hypodermic needle
S40-S43 method actions

1. A heat transfer device which is provided to be brought into contact with a hand of a human and which is arranged to transfer heat to, or from, the hand, the hand being attached to the end of an arm at a wrist and having a first main side and a second main side located opposite the first main side, the first main side comprising the opisthenar area of the hand and the second main side comprising the palm of the hand, wherein a distal direction of the hand is defined as a direction towards the fingertips of the hand, and a proximal direction of the hand is defined as a direction towards a wrist, the distal direction and the proximal direction together defining a longitudinal direction, and a direction perpendicular to the longitudinal direction and generally along the surface of the hand being a widthwise direction,
wherein the heat transfer device is for use with a heat transfer fluid arranged to flow through the heat transfer device,
wherein the heat transfer device comprises a fluid chamber through which the heat transfer fluid is arranged to flow;
wherein the heat transfer device comprises a front portion which is arranged to cover at least a portion of the fingers of the hand on the first main side of the hand,
wherein the heat transfer device comprises a rear portion which extends in the proximal direction from the front portion, wherein the rear portion has an opening in the region of the opisthenar area so as to leave at least a portion of the opisthenar area uncovered.

2. The heat transfer device of claim 1, wherein the opening has a size, wherein the size is:
such that the portion of the opisthenar area left uncovered is large enough to accommodate a hypodermic needle, preferably also an adhesive bandage for temporarily fixing the hypodermic needle to the hand, and/or
such that the portion of the opisthenar area left uncovered has at least a minimum width in the widthwise direction, wherein the minimum width is at least 3 mm, 5 mm, 1 cm, 2 cm, 3 cm or 4 cm, and/or such that the portion of the opisthenar area left uncovered has at most a maximum width in the widthwise direction, wherein the maximum width is at most 6 cm, 5 cm, 4 cm, 3 cm or 2 cm.

3. The heat transfer device of claim 1, wherein the opening is open towards the proximal direction.

4. The heat transfer device of claim 1, wherein the heat transfer device does not have a cover for covering the opening.

5. The heat transfer device of claim 1, wherein the rear portion comprises at least one lateral portion which is arranged to cover a portion of the opisthenar area, wherein the lateral portion is adjacent to the opening in the widthwise direction.

6. The heat transfer device of claim 1, wherein the front portion is arranged to cover substantially the entire finger region on the first side of the hand.

7. The heat transfer device of any one of claim 1, wherein the heat transfer device is arranged to cover at least a portion of the hand on the second side, in particular at least 50%, 60%, 70%, 80% or 90%, in particular substantially all of the hand on the second side.

8. The heat transfer device of claim 1, wherein, on the second side and in the proximal direction, the heat transfer device is arranged to extend beyond the wrist.

9. The heat transfer device of claim 1, wherein the heat transfer device further comprises a member arranged to extend around the arm to secure the heat transfer device to the arm.

10. The heat transfer device of claim 9, wherein the member is at least partially see-through.

11. The heat transfer device of claim 1, wherein the heat transfer device has at least a first fluid connection to enable the heat transfer fluid to enter the fluid chamber and at least a second fluid connection to enable the heat transfer fluid to exit the fluid chamber.

12. Apparatus for use in the thermal therapy treatment of a human hand, in particular for use in the treatment of tissue damage after surgical interventions, of injuries, of inflammations and of chronic diseases of the rheumatic type, and in particular for assisting chemotherapeutic treatments, wherein the apparatus comprises:
a temperature control device which is provided in order to control a temperature of a heat transfer fluid; and
at least one heat transfer device according to claim 1,
wherein the temperature control device is connected, in a fluid conducting manner, to the at least one heat transfer device, and is further arranged to temper the at least one heat transfer device with the heat transfer fluid flowing through the heat transfer device.

13. A method of using the heat transfer device of claim 1 for thermal therapy treatment of a hand of a human, wherein the method comprises:
applying the heat transfer device to a hand of a human; and
causing the heat transfer fluid to flow through the heat transfer device.

14. A method of using the apparatus of claim 12 for thermal therapy treatment of a hand of a human, wherein the method comprises:
applying the heat transfer device to a hand of a human; and
causing the heat transfer fluid to flow through the heat transfer device.
